(19) United States
(12) Patent Application Publication
(10) Pub. No.: US 2008/0226612 A1
(43) Pub. Date: Sep. 18, 2008

(54) Compositions of Cells Enriched for Combinations of Various Stem and Progenitor Cell Populations, Methods of Use Thereof and Methods of Private Banking Thereof

(75) Inventors: Avraham Treves, Mevaseret Zion (IL); Ronald Hoffman, Chicago, IL (US); Arnon Nagler, Jerusalem (IL); Ami Treves, Minneapolis, MN (US)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Bio Regenerate, Inc., Minneapolis (IL)

(21) Appl. No.: 12/064,034

(22) PCT Filed: Aug. 2, 2006
(86) PCT No.: PCT/US06/30389

§ 371 Date: Feb. 18, 2008

Related U.S. Application Data
(60) Provisional application No. 60/709,537, filed on Aug. 19, 2005.

(51) Int. Cl. C12N 5/06; A61K 48/00; A61K 35/14; A61P 19/00; A61P 25/00; A61P 17/00; A61P 17/02; A61P 1/16; A61P 9/00; A61P 7/06

(52) U.S. Cl. 424/93.21; 435/347; 435/395; 435/374; 424/93.7

(57) ABSTRACT

The present disclosure covers compositions and method for the preparation and use of mixtures of adult stem/progenitor cell populations recovered and enriched from specific tissues with very limited attempts for their purification. Such mixtures of cell populations have improved therapeutic effectiveness in the treatment of certain diseases and tissue regeneration treatments over their more purified counterpart cell populations. Such mixtures of cell populations can be cryopreserved for future clinical use.

### BACKGROUND

Cell therapy, the use of living cells as therapeutic agents, is a medical approach presently being used for several clinical indications such as treatment of injured joints, chronic ulcers, corneal damage, large burns, neural damage and others. A unique population of cells, stem cells (SC), are of special interest due to their self-renewal capacity and their potential to differentiate and develop into several different cell lineages.

There are two major types of stem cells. Embryonic stem cells (ES) are derived from blastocysts which arise in a very early stage of embryonic development. ES cells can be grown in culture to large numbers but are difficult to control in their development and are accompanied by unresolved ethical problems. A second type of stem cell is the adult stem cell (ASC), which is found in various tissues of the adult body. Each tissue and organ in the body originates from a small number of ASCs which are committed to differentiate into the various cell types that compose the tissue. ASCs are a likely source of continuous normal tissue replenishment as well as recovery in case of damage or disease throughout the life of the organism.

The first and most widely studied tissue in animals is the blood. Most if not all, blood cells, including red blood cells, lymphocytes, monocytes, polymorphs, and platelets originate from a population of stem cells known as hematopoietic stem cells (HSCs) which are located in the bone marrow, in the circulation and other organs.

HSCs from either bone marrow, peripheral blood or cord blood, are widely used for replacement of ablated bone marrow and treatment of malignant and genetic diseases. In addition to HSCs, it was recently found that bone marrow contains primitive stem cells that can differentiate into other tissues and organs. Some of the ASC in the bone marrow are part of a well characterized population of stem cells known as mesenchymal stem cells that can differentiate into bone, cartilage and heart muscle cells but other pluripotent stem cells have also been detected. ASCs have been isolated recently from cord blood, adult peripheral blood, fat tissue and other organs. Under suitable conditions they can give rise to additional tissues such as blood vessels, bone, cartilage, muscle, liver, nerve cells as well as insulin secreting Langerhans cells.

Additional types or populations of ASCs have also been identified in various tissues. Actually, every tissue and organ in the body is likely to contain stem cells that participate in intrinsic regeneration and repair during growth, trauma and disease.

Mesenchymal stem cells have been described in adult human bone marrow. Human bone marrow has been reported to be a source of pluripotent stem cells, in addition to the hematopoietic stem cells. Bone marrow derived hematopoietic stem cells were also reported to maintain pluripotent potential for non-hematopoietic tissues. Hematopoietic stem cells with pluripotent potential have also been found in other tissues such as cord blood.

Various types of hematopoietic and non-hematopoietic stem and progenitor cells have also been found in human blood. Populations of endothelial progenitor cells, mesenchymal stem cells as well as fibrocytes that can mediate tissue repair have all been reported.

Recently, monocyte and macrophage like cell populations that express pluripotent potential were reported to reside in the circulation of healthy adult people. Peripheral blood endothelial progenitor cells that secrete angiogenic growth factor, have also been reported to be derived from populations of monocyte/macrophages. Populations of non-hematopoietic pluripotent stem cells have been reported in non-mobilized human peripheral blood.

Each of these stem cells from these various sources are being tested clinically for treatment of diseases such as ischemic heart, neural injuries, neuro-degenerative diseases, diabetes, as well as other diseases that do not currently have effective treatments. Many additional disease indications are under investigation at their pre-clinical research stage. Currently however, major limitations to the use of adult stem cells include their scarce availability in adults and histological barriers between individuals that may restrict their transplantation. To improve availability, several approaches have recently been developed that can be used to generate stem cells from bone marrow and cord blood in sufficient numbers for therapeutic use. Several types of pluripotent stem and progenitor cells have also been identified recently in normal adult peripheral blood. Methods for isolating these cells are based on their membrane markers and plastic adherence properties. Methods are also described for their ex vivo expansion. However, it remains unclear which cell population is responsible for each in vivo function, and in several cases, therapeutic activity of defined stem cell populations was not demonstrated and the origin of the therapeutic cells is controversial.

The isolation and purification of a defined population of stem cells in some instances yields cell populations that are of limited clinical use. In addition, technical difficulties in their isolation and storage are commonly encountered. Moreover, the art of stem cell science is relatively new and undeveloped and defined populations of cells may have limited clinical potential.

### SUMMARY

The present disclosure covers compositions and methods for the preparation and use of enriched populations of adult stem and progenitor cells isolated from specific tissues. The cell populations are obtained with very limited attempts for their purification and enriched for most of the populations of stem and progenitor cells which are found in the original tissue. The populations have improved therapeutic effectiveness in the treatment of diseases and tissue regeneration treatments over their more purified counterpart cell populations.

To this end, the present application is directed to mixtures of various stem and progenitor cell populations obtained from the body tissues where they are found, their method of extraction, their preservation and clinical utilization. In particular methods and embodiments, the tissue can be blood, placenta, ascetic fluid, skin, kidney, liver, muscle, neural tissue or fat tissue. Generally, the tissue is not umbilical cord tissue. Preferably, the tissue is unmobilized peripheral blood.

The present disclosure also covers the business process of extracting mixtures of various stem and progenitor cell populations from un-mobilized peripheral blood or other tissues and their private storage for individuals' future medical needs as well as for clinical use by other individuals and deriving revenue from the extraction and storage of these cell populations. Such mixtures of various cell populations, which are not purified, are more effective and more practical for use in clinical applications.

Additional features and advantages are described herein, and will be apparent from, the following Detailed Description.

### BRIEF DESCRIPTION OF THE FIGURE

FIG. 1 is a graphical representation of the proliferative capacity of cells produced using the double adherence method described herein.

### DETAILED DESCRIPTION

The present invention is directed to enriched and unpurified mixtures of populations of stem cells and progenitor cells and their use as therapeutic agents. The mixtures include populations of cells recovered from suitable body tissues and can include adult stem cells and progenitor cells. The recovery methods while being sufficient to obtain the desired cells from tissues, will generally not include further purification. Thus, the recovered cells in the cell populations and mixtures generally will include the substantially all and more preferably all populations of stem and progenitor cells in the original tissue, with a reduced amount of mature lymphoid and myeloid cells.

For present purposes the phrase “cell populations” refers to populations such as hematopoietic stem and progenitor cells, mesenchymal stem and progenitor cells, monocytic derived stem and progenitor cells, stromal derived stem and progenitor cells, endothelial progenitor cells, multipotent adult progenitor cells, pluripotent adult stem cells and the like. Mixtures of cell populations is meant to refer to mixtures of such populations.

The cell mixture can be prepared from tissues isolated from relatively young and healthy individuals, or from individuals at risk for certain diseases, or from individuals with certain diseases, or suspected to carry certain diseases, so that, when needed, the stem and progenitor cell populations will be autologous and readily available, and thereby avoid histocompatibility and immune-suppression processes.

In certain methods and embodiments, subpopulations of the isolated cells can be used in allogeneic transplantation therapies. For example, subpopulations of cells that are generally lower in cell markers could be used. In such cases it can be necessary to produce large batches of therapeutic cell preparations.

In certain methods and embodiments, the recovered cell mixtures can be separated into more defined and purified cell populations for defined clinical applications.

Suitable tissues for use in generating the cell populations include all those tissues which harbor the desired cells. For example, suitable tissues can include blood, cord blood, cord matrix, blood buffy coat, placenta, amniotic fluid, ascitic fluid, skin, kidney, liver, muscle, neural tissue, fat, tooth pulp, and the like. Generally however, the tissue will not be umbilical cord tissue.

Methods for mobilizing stem and progenitor cells into the blood, particularly from bone, are well known in the art and are generally avoided and not used in certain of the methods. Thus, in a preferred embodiment, mixtures of stem and progenitor cells are recovered from unmobilized blood tissue.

Cell populations can be recovered by extraction. Many extraction methods are known in the art and can be used, so long as they can be used to obtain the described mixtures of stem and progenitor cells from the bulk of the ancillary tissue components including one or more of the following red cells, platelets, granulocytes, unwanted fluids, and tissue matrix. Suitable cell extraction methods include one or more of the following known methods: plasmapheresis, centrifugation at defined time and g-force or density gradient centrifugation, centrifugation following the addition of some fluids such as physiological solutions or certain soluble polymers, cellular adherence to plastic, and adherence to reagents used to coat growth surfaces including reagents such as fibronectin, and collagen. In addition, mechanical cell sorting methods can be used and enzymatic methods can be used, as are known. The preferred method for recovery would be either centrifugation or plastic adherence or combinations of both methods. The centrifugation can be done either directly in the blood collection bag, or following introduction of certain fluids, or following the transfer of the fluid to another container.

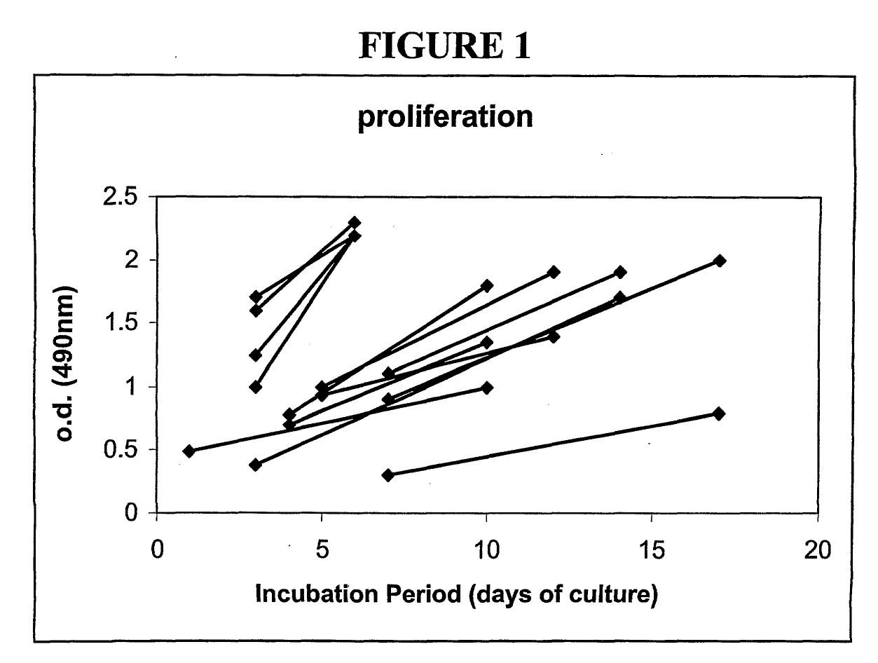

In one method, blood buffy coat can be obtained from a unit of peripheral blood using a standard centrifugation process of the blood bag that is more commonly used to remove the bulk of white blood cells. Alternatively, a more enriched buffy coat fraction can be obtained by modifying the velocity and time of centrifugation, and/or adding fluids that would change sedimentation rate. The white blood cell fraction is then separated on ficoll layer to enrich for mononuclear cells and remove platelets, granulocytes and erythrocytes, for example by centrifugation for 30 min at 800×g. The cells obtained can be suspended in culture medium such as D-MEM low glucose, containing the following cytokines: M-CSF 25 ng/ml., LIF 1000 units/ml (10 ng/ml ), IL-6 20 ng/ml., FGF-beta 10 ng/ml. The cell suspensions can then be plated in T75 tissue culture flasks at a concentration of 4×10⁶ per ml. Flasks can be pre-coated with fibronectin, by pre-incubation for 24 h with 8 ml solution of 10 μl/ml of fibronectin in PBS. After about 4-5 days, the non-adherent cells are recovered and re-plated under the same conditions in additional T75 flasks. Culture medium is added to the adherent fraction (first round adherent cells). After 4-10 additional days, the non-adherent cells from both first and second round adherent fractions are removed, and the adherent cells are recovered from both fractions by incubation for 5 min with trypsin-EDTA solution. The cells from each adherent fraction can be tested for markers and stored separately, or combined and stored or used as desired. This method enables recovery of maximal numbers and types of stem and progenitor cells as well as insures reproducibility among individual blood donors. A representative table of numbers of cells recovered by this method is given in Table 1. The cell morphologies represented in the mixture include spindle shape, mesenchymal like and endothelial like, and monocytes and macrophages. The cells from the non-adherent fractions can be separated on affinity columns of CD34 or CD133 positive beads, to recover hematopoietic stem and progenitor cells. Phenotypic analysis of the adherent fractions is shown in Table 2. The cells recovered by this method maintain some proliferative capacity under these culture conditions as shown in FIG. 1.

TABLE 1
Recovery of adherent stem/progenitor populations from different healthy people

| | | No of adherent | no of adherent | |
|---|---|---|---|---|
| | | cells 1^{st} | cells 2^{nd} | Total adherent |
| Exp. Number | No. of MNCs | fraction | fraction | cells recovered |
| 28 | 400 × 10⁶ | 0.94 × 10^{6(15 d)} | 5 × 10^{6(15 d)} | 5.94 × 10⁶ |
| 30 | 225 × 10⁶ | 1.7 × 10^{6(12 d)} | 6 × 10^{6(12 d)} | 7.7 × 10⁶ |
| 31 | 400 × 10⁶ | 17 × 10^{6(5 d)} | 21 × 10^{6(12 d)} | 38 × 10⁶ |
| 32 | 420 × 10⁶ | 0.74 × 10^{6(13 d)} | 15 × 10^{6(13 d)} | 15.7 × 10⁶ |
| 33 | 400 × 10⁶ | 3.4 × 10^{6(10 d)} | 10 × 10^{6(10 d)} | 13.4 × 10⁶ |
| 34 | 405 × 10⁶ | 1.8 × 10^{6(8 d)} | 10 × 10^{6(8 d)} | 11.8 × 10⁶ |
| Mean ± S.E | 375 ± 74 × 10⁶ | 4.3 ± 6.3 × 10⁶ | 11.2 ± 6.0 × 10⁶ | 15.4 ± 11.6 × 10⁶ |

(#d indicates the number of days in culture)

TABLE 2
Phenotype characterization of adherent cells

| | | | | |
|---|---|---|---|---|
| | Exp No 32 | Exp No 34 | Exp No 32 | Exp No 28 |
| Marker | (5 d) | (8 d) | (13 d) | (15 d) |

Adherent 2nd fraction

| | | | | |
|---|---|---|---|---|
| CD105 | 46-89% | 80-86% | | 90% |
| CD31 | 6-24& | 29-31% | 80-87% | 30-80% |
| VE Cadherin | 5-10% | 8-12% | 28% | 58% |
| VEGF R2 | 25% | 6% | 9% | 5% |
| CD90 | 12% | 7-10% | 19-26% | 41-50% |
| CD45 | ND | 90% | 98% | 90% |

Adherent 1st fraction

| | | | | |
|---|---|---|---|---|
| CD105 | 85% | 82% | ND | 96% |
| CD31 | 18-23% | 14-36% | 74% | 70-76% |
| VE Cadherin | 4% | ND | ND | ND |
| VEGF R2 | 7% | 8% | ND | 42% |
| CD90 | 0-3% | 1-8% | 39% | 48-53% |
| CD45 | 93% | ND | 99% | ND |

The % ranges for each marker are from different measurements with different reference markers.

Adherent cells were generated by the method described above and were cultured for different time periods (2-7 days) and for different cell passages (p0, p1, p2 or p3). Cells (at concentrations of 2.5×10⁴−4×10⁶/ml) were plated in 96 well plates and examined after different incubation periods for cell proliferation by the EZ4U, modified MTT test, Growth curves of 14 independent cultures are demonstrated. The two curves labeled “F” represent cell cultures grown on fibronectin pre-coated wells,

In an embodiment, the method for isolating suitable cell populations includes obtaining a fluid from an animal or human tissue source, incubating the fluid on an attachment media for a period of time ranging from about 2 h to about three days or more, separating the non-adherent cells from the adherent cells using known methods. For example, cells can be grown in flasks which can be coated with fibronectin or collagen or other suitable coating agent and the supernatant containing the non-adherent cells, and incubating the adherent cells in culture media for a period of time ranging from about 1 day to about 1 week or more to obtain a suitable cell population. Culture medium used with the cells can include serum and growth factors, as required. Once obtained, an adhered cell population can be placed in a suitable storage media and stored. In an embodiment, storage can include the use of low temperatures in a cryopreservation method.

In an embodiment, the autologous plasma will also be recovered and stored or used for the culture or for the separation procedure, thus avoiding the need for foreign serum.

In an embodiment, the isolated mixture of cell population will have the following surface markers, which need not be expressed on a single cell but rather can be expressed on any of the cells in the population, so long as the population as a whole includes a variety of markers from the following group: CD11, CD14, CD31, CD34, CD44, CD45, CD90, CD102, CD117, CD133, CD135, CD166, CXCR4, c-met, Mac-1, c-kit, SH-2, SH3, SH4, VE-Cadherin, VEGFR, VWF, and Tie-2s. In an embodiment the cell mixture will contain at least 20 of the listed markers. In other embodiments the cell mixtures will contain at least 19 or 18, 17, 16, 15, 14, 13, 12, 11, 10, 9, 8, 7, 6, 5 or 4 of the listed markers.

In an embodiment, the cells are not activated ex vivo.

In certain embodiments, the cell mixture contains hematopoietic cells, or hematopoietic committed cell lineages including lymphoid cells, erythroid cells, myeloid cells, monocytic cells, megakaryocytic cells and the like, including their combinations or combinations with other stem and progenitor cell populations.

In certain embodiments, the cell populations include hematopoietic cells, hematopoietic committed cell lineages, mesenchymal stem cells, stromal cells, fibroblasts, endothelial progenitor cells and the like and their mixtures.

The present disclosure also contemplates the use of mixtures of cells as therapeutic cell populations and as therapeutic agents. To this end, a method is disclosed that includes obtaining a fluid from an animal or human tissue source, incubating the fluid on an attachment media for a period of time ranging from about 2 h to about three days or more, separating the non-adherent cells from the adherent cells, and treating a patient having a disease with a portion of the cells. In a variation of the disclosed method the adherent cells can be incubated in culture media for a period of time ranging from about 1 day or more to about 4 weeks. In a method, the mixture of cells will be obtained by centrifugation procedures of the blood or body fluid. In a method, the cells can be preserved or stored until use by suitable preservation or storage methods. Preservation methods and storage methods are known in the art and can be used so long as the various populations of cells in the preserved sample are not substantially changed. For example, one suitable method is a cryopreservation method, as is known. A patient can then be treated with cells derived from the cryopreserved cells after their thawing.

Cells can be administered to patients by any method that allows the cells to reach the sites needed for the composition to generate the desired therapeutic effect. For example, cells can be administered by intravenous injection or by injection directly into specific organs, or directly to the site of action.

Diseases that can be treated by the present methods include those that can be treated by tissue regeneration, by protein replacement, or by coagulation factors. Such diseases include diseases associated with defective biological processes such as cardiac ischemia, osteoporosis, chronic wounds, diabetes, neural degenerative diseases, neural injuries, bone or cartilage injuries, ablated bone marrow, anemia, liver diseases, hair growth, teeth growth, retinal disease or injuries, ear diseases or injury, muscle degeneration or injury, plastic surgery. In addition, the treatment methods can be applied to cosmetic therapies including, filling of skin wrinkles, supporting organs, supporting surgical procedures, treating burns, and treating wounds, for example.

Specific treatment methods can include situations in which the combination between the donor and recipient of the cells is either autologous or allogeneic.

The present application also encompasses methods for preparing a therapeutic agent containing a product secreted from the aforementioned cell populations. To this end, the method can be accomplished by preparing a cell population by any of the methods described previously and incubating the cells in culture media for a period of time sufficient to generate secreted products. The secreted products can then be isolated from the culture media by known methods which one of skill in the art can appreciate will depend upon the nature of the product.

The present application further encompasses methods of using the disclosed cell populations in gene therapy. Such methods can be accomplished by preparing cell populations by methods as described above. The mixture of cells can then be transfected with a recombinant DNA or other methods of gene manipulation to modify the cell genetics and the modified cells can be introduced into a patient in need thereof or used to generate product which can be administered to a patient. Numerous recombinant techniques and recombinant DNAs that are useful for modifying the genetics of stem and progenitor cells are known in the art and can be used.

The present application further encompasses methods for generating revenues for a business that utilize the above disclosed compositions and processes. To this end, an amount of a mixture of cell populations can be obtained from a body tissue such as by the methods disclosed above and the mixture of cell population can be placed into a storage device and stored. The cell population can be dispensed and provided to a patient in need of the cells. A fee can be charged for the isolation, storage and/or dispensing of the cells to generate a business revenue. In a further method a tissue sample, once obtained from an individual, can be transported to a central location and the mixture of cell populations can be extracted from tissue at the central location. In yet a further method, the mixture of cell populations can be stored at a central location, such as by cryopreservation.

In a further method, the cells from the original tissue such as blood or buffy coat or mononuclear cells can be stored before enrichment of the stem and progenitor populations. Before use, the cells are thawed and the extraction method described above is applied.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A therapeutic agent comprising: an isolated population of cells comprising adult stem cells and progenitor cells isolated from tissue other than the umbilical cord by a method that consists essentially of extraction.

2. The therapeutic agent of claim 1, wherein substantially all populations of stem and progenitor cells found in the tissue source are in the isolated population of adult stem cells and progenitor cells.

3. The therapeutic agent of claim 1 wherein the tissue includes a tissue selected from the group of tissues consisting of blood, unmobilized peripheral blood, placenta, amniotic fluid, ascitic fluid, skin, kidney, liver, muscle, and neural tissue.

4. The therapeutic agent of claim 1 wherein the extraction method removes one or more of the bulk of red cells, platelets, granulocytes, unwanted fluids and tissue matrix.

5. The therapeutic agent of claim 1 wherein the extraction method includes plasmapheresis.

6. The therapeutic agent of claim 1 wherein the extraction method includes density gradient centrifugation.

7. The therapeutic agent of claim 1 wherein the extraction method includes plastic adherence.

8. The therapeutic agent of claim 1 wherein the extraction method includes adherence to a coating reagent.

9. The therapeutic agent of claim 1 wherein the extraction method includes adherence to the coating reagent fibronectin.

10. The therapeutic agent of claim 1 wherein the extraction method includes adherence to a coating reagent collagen.

11. The therapeutic agent of claim 1 wherein the extraction method includes a mechanical method.

12. The therapeutic agent of claim 1 wherein the extraction method includes an enzymatic method.

13. The therapeutic agent of claim 1 wherein the population of cells is isolated from blood buffy coat.

14. The therapeutic agent of claim 1 wherein the population of cells is isolated from a plasmapheresis product.

15. A method for isolating a population of cells comprising adult stem cells and progenitor cells from animal tissue comprising:
obtaining a fluid comprising cells from a nonumbilical cord tissue source;
incubating the fluid on an attachment media for a period of time ranging from about 2 h to about three days;
separating the non-adherent cells from the adherent cells;
incubating the adherent cells in culture media for a period of time ranging from about 1 day or more to about 4 weeks.

16. The method of claim 15 wherein the culture media comprises serum.

17. A method for preserving a mixture of cells comprising isolating a population of cells comprising adult stem cells and progenitor cells from a nonumbilical cord tissue substantially without further purification and cryopreserving the cells.

18. A composition of cells comprising: a population of adult stem cells and progenitor cells wherein the cell population comprises at least four surface markers selected from the group of surface markers consisting of CD11, CD14, CD31, CD34, CD44, CD45, CD90, CD102, CD117, CD133, CD135, CD166, CXCR4, c-met, Mac-1, c-kit, SH-2, SH3, SH4, VE-Cadherin, VEGFR, VWF, and Tie-2s.

19. The composition of cells of claim 18, wherein the population comprises at least five surface markers selected from the group of surface markers consisting of CD11, CD14, CD31, CD34, CD44, CD45, CD90, CD102, CD117, CD133, CD135, CD166, CXCR4, c-met, Mac-1, c-kit, SH-2, SH3, SH4, VE-Cadherin, VEGFR, VWF, and Tie-2s.

20. The composition of cells of claim 18, wherein the population comprises at least ten surface markers selected from the group of surface markers consisting of CD11, CD14, CD31, CD34, CD44, CD45, CD90, CD102, CD117, CD133, CD135, CD166, CXCR4, c-met, Mac-1, c-kit, SH-2, SH3, SH4, VE-Cadherin, VEGFR, VWF, and Tie-2s.

21. The composition of cells of claim 18, wherein the population comprises at least fifteen surface markers selected from the group of surface markers consisting of CD11, CD14, CD31, CD34, CD44, CD45, CD90, CD102, CD117, CD133, CD135, CD166, CXCR4, c-met, Mac-1, c-kit, SH-2, SH3, SH4, VE-Cadherin, VEGFR, VWF, and Tie-2s.

22. The composition of cells of claim 18, wherein the population comprises at least twenty surface markers selected from the group of surface markers consisting of CD11, CD14, CD31, CD34, CD44, CD45, CD90, CD102, CD117, CD133, CD135, CD166, CXCR4, c-met, Mac-1, c-kit, SH-2, SH3, SH4, VE-Cadherin, VEGFR, VWF, and Tie-2s.

23. The composition of cells of claim 18, wherein the cells are not activated ex vivo.

24. The composition of cells of claim 18 wherein the cell population comprises hematopoietic cells.

25. The composition of cells of claim 18 wherein the cell population comprises cells having hematopoietic committed lineages.

26. The composition of cells of claim 18 wherein the cell population comprises cells having hematopoietic committed lineages comprising lymphoid cells.

27. The composition of cells of claim 18 wherein the cell population comprises cells having hematopoietic committed lineages comprising erythroid cells.

28. The composition of cells of claim 18 wherein the cell population comprises cells having hematopoietic committed lineages comprising myeloid cells.

29. The composition of cells of claim 18 wherein the cell population comprises cells having hematopoietic committed lineages comprising monocytic cells.

30. The composition of cells of claim 18 wherein the cell population comprises cells having hematopoietic committed lineages comprising megakaryocytic cells.

31. The composition of cells of claim 18 wherein the cell population comprises mesenchymal cells.

32. The composition of cells of claim 18 wherein the cell population comprises stromal cells.

33. The composition of cells of claim 18 wherein the cell population comprises fibroblasts.

34. The composition of cells of claim 18 wherein the cell population comprises endothelial progenitor cells.

35. A method for treating a patient with a cell-based therapeutic agent comprising:
obtaining a fluid comprising cells from an a nonumbilical cord tissue source;
incubating the fluid on an attachment media for a period of time ranging from about 2 h to about three days;
separating the non-adherent cells from the adherent cells;
treating a patient having a disease with at least a portion of the cells,

36. The method for treating a patient with a cell-based therapeutic agent of claim 35 further comprising:
incubating the adherent cells in culture media for a period of time ranging from about 1 day or more to about 4 weeks.

37. A method for treating a patient with a cell-based therapeutic agent comprising:
obtaining a fluid comprising cells from a nonumbilical cord tissue source;
incubating the fluid on an attachment media for a period of time ranging from about 2 h to about three days;
separating the non-adherent cells from the adherent cells;
incubating the adherent cells in culture media for a period of time ranging from about 1 day or more to about 4 weeks;
cryopreserving the cultured cells;
treating a patient having a disease with the cryopreserved cells.

38. The method for treating a patient with a cell-based therapeutic agent of claim 37 wherein the treatment includes intravenous injection of the cells.

39. The method for treating a patient with a cell-based therapeutic agent of claim 37 wherein the treatment includes injection of the cells directly into specific organs.

40. The method for treating a patient with a cell-based therapeutic agent of claim 37 wherein the disease can be treated by tissue regeneration.

41. The method for treating a patient with a cell-based therapeutic agent of claim 37 wherein the disease can be treated by protein replacement.

42. The method for treating a patient with a cell-based therapeutic agent of claim 37 wherein the disease can be treated by coagulation factors.

43. The method for treating a patient with a cell-based therapeutic agent of claim 37 wherein the disease is associated with biological processes selected from the group of processes consisting of cardiac ischemia, osteoporosis, chronic wounds, diabetes, neural degenerative diseases, neural injuries, bone or cartilage injuries, ablated bone marrow, anemia, liver diseases, hair growth, teeth growth, retinal disease or injuries, eye diseases or injuries, ear injuries or diseases muscle degeneration or injury.

44. The method for treating a patient with a cell-based therapeutic agent of claim 37 wherein the patient is in need of a cosmetic therapy selected from the group of cosmetic therapies consisting of filling of skin wrinkles, supporting organs, supporting surgical procedures, treating burns, and treating wounds.

45. The method for treating a patient with a cell-based therapeutic agent of claim 37 wherein the combination between the donor and recipient of the cells is autologous.

46. The method for treating a patient with a cell-based therapeutic agent of claim 37 wherein the combination between the donor and recipient of the cells is allogeneic.

47. A method for preparing a therapeutic agent comprising:
obtaining a fluid that includes cells from a nonumbilical cord tissue source;
incubating the fluid on an attachment media for a period of time ranging from about 2 h to about three days;
separating the non-adherent cells from the adherent cells;
incubating the adherent cells in culture media for a period of time sufficient to generate secreted products;
collecting the tissue culture fluid and isolating a secreted product from the culture media.

48. A gene therapy method comprising:
obtaining a fluid that includes cells from a nonumbilical cord tissue source;
incubating the fluid on an attachment media for a period of time ranging from about 2 h to about three days;
separating the non-adherent cells from the adherent cells;
incubating the adherent cells in culture media for a period of time sufficient to generate secreted products;
modifying the genetics of an adherent cell using a genetic manipulation and
introducing the modified cell into a patient in need thereof.

49. A method of generating revenue comprising:
obtaining an amount of stem and progenitor cells from a tissue other than the umbilical cord,
placing the cells into a storage device,
storing the cells,
dispensing the stem cells to a patient in need of the stem cells,
charging a fee.

50. The method of generating revenue of claim 49 wherein the fee is charged for obtaining the stem and progenitor cells.

51. The method of generating revenue of claim 49 wherein the fee is charged for storing the stem and progenitor cells.

52. The method of generating revenue of claim 49 wherein the fee is charged for dispensing the stem and progenitor cells.

53. A method of generating revenue comprising:
obtaining an amount of a body tissue other than umbilical cord tissue from an individual,
transporting the tissue to a central location;
processing the tissue by extracting the stem cell/progenitor cell population from the tissue unit;
storing the extracted stem cell/progenitor cell population at the central location
dispensing the stem cells to a patient in need of the stem cells,
charging a fee.

54. A method of generating revenue of claim 53 wherein the step for storing the stem cell/progenitor cell population comprises cryopreservation.

55. A therapeutic agent comprising: an isolated population of cells comprising adult stem cells and progenitor cells isolated from unmobilized peripheral blood by a method that consists essentially of extraction.
